(19) United States
(12) Patent Application Publication
(10) Pub. No.: US 2016/0143572 A1
(43) Pub. Date: May 26, 2016

(54) INCONTINENCE MONITORING DEVICE

(71) Applicant: Timea SUSNJAR, Schaumburg, IL (US)

(72) Inventor: Timea SUSNJAR, Schaumburg, IL (US)

(21) Appl. No.: 14/731,408

(22) Filed: Jun. 5, 2015

(30) Foreign Application Priority Data
Nov. 21, 2014 (GB) 1420779.9

(51) Int. Cl. A61B 5/20; G08C 19/00; G08B 5/22; A61B 5/00; G08B 21/18

(52) U.S. Cl. CPC A61B 5/202; A61B 5/0004; G08B 21/182; G08B 5/22; G08C 19/00

(57) ABSTRACT

An incontinence monitoring device has a face separated into substantially parallel upper and lower surfaces having a flexible material held therebetween. The device includes a means of registering excessive moisture levels with respect to at least one of the surfaces and a system for conveying information regarding the registering of excessive moisture levels to remote users.

### CROSS REFERENCE TO EARLIER APPLICATIONS

This application claims priority under 35 U.S.C. §119 to GB patent application no. 1420779.9 filed Nov. 21, 2014, the disclosure of which is incorporated herein by reference in its entirety.

### TECHNICAL FIELD

The present invention relates to an incontinence monitoring device, in particular a device for monitoring of incidents of incontinence remotely; more particularly but not exclusively a means of providing a comfortable and discrete seat that notifies remote users of local incontinence.

### BACKGROUND

Increasing ageing of populations in many societies has led to an increased need for monitoring of vulnerable persons or patients. Such monitoring can be time-consuming and difficult.

In particular for patients who are particularly vulnerable to incontinence, including children or handicapped individuals, all collectively herein known as patients, there can be frequent moments of distress from incontinence if monitoring is not constant.

### PRIOR ART

Accordingly a number of patent applications have been filed in an attempt to resolve the problem or similar, including the following:

Granted U.S. Pat. No. 8,383,876 (ROBLES et al) discloses an absorbent article, characterized in that the article comprises: a first indicating zone with at least a portion of each of a first number of visual fullness indicating areas; and a second indicating zone with at least a portion of each of a second number of visual fullness indicating areas, wherein: the second indicating zone is immediately adjacent to the first indicating zone, the second indicating zone is outboard from the first indicating zone, an overall width of the second indicating zone is equal to an overall width of the first indicating zone, and the second number differs from the first number.
Granted U.S. Pat. No. 6,583,722 (JEUTTER et al) discloses an article having a monitored portion, and a status signaling device for communicating a change in status of said monitored portion, said status signaling device comprising: a sensor which can indicate a change of state that occurs when said monitored portion of said article changes from a first condition to a different, second condition; and a passive electronic transponder which is cooperatively connected with said sensor, wherein said transponder is configured to receive electromagnetic input energy which has been provided to said transponder at a predetermined input frequency, and said transponder is configured to respond with operative indicator data when said monitored portion is in said second condition.
Granted U.S. Pat. No. 6,284,942 (RABIN) discloses a pad which permits a quantitative estimate of urinary incontinence, said pad comprising: (A) a first zone having a defined urine absorption capacity and undergoing a first color change as it becomes wetted with urine; (B) a second zone having a urine absorption capacity greater than the capacity of said core zone and undergoing a second color change when it becomes wetted with urine; and (C) a third zone having a urine absorption capacity greater than the capacity of said inner zone and undergoing a third color change when it becomes wetted with urine; each of said first, second and third color changes being visually distinct.

### SUMMARY

According to the present invention there is provided an incontinence monitoring device comprising a face separated into substantially parallel upper and lower surfaces having a flexible material held therebetween, a means of registering excessive moisture levels with respect to at least one of the surfaces and a system for conveying information regarding the registering of excessive moisture levels to remote users.

In this way the device does not cause distress to a patient, wherein remote users may be alerted without local disturbance.

According to a second aspect of the present invention therefore there is provided a system of monitoring incontinence incorporating a device substantially as described herein incorporated into furniture, bedclothing or garments.

According to another aspect, a device is provided comprising: a face separated into substantially parallel upper and lower surfaces; means of detecting a presence of fluid with respect to at least one of the surfaces; and means for conveying information regarding the registering of excessive moisture levels to remote users.

According to another aspect, a device is provided comprising: an upper surface and a lower surface that cooperate to form an enclosure, wherein the upper surfaces is at least partially fluid permeable; a moisture-sensing circuit disposed within the enclosure; and a module communicatively coupled with the moisture-sensing circuit, the moisture-sensing circuit outputting a signal to a remove user when fluid passes through or contacts the upper surface, and contacts the moisture-sensing circuit.

According to another aspect, a device is provided comprising: an upper surface and a lower surface that cooperate to form an enclosure, wherein the upper surfaces is at least partially fluid permeable; a moisture-sensing circuit disposed within the enclosure; and a module communicatively coupled with the moisture-sensing circuit, the moisture-sensing circuit outputting a signal to a remove user when fluid passes through or contacts the upper surface, and contacts the moisture-sensing circuit; and a computing device communicatively coupled with the device over a network, the computing device being configured to monitoring fluid levels on the upper surface of the device.

### BRIEF DESCRIPTION OF FIGURES

FIG. 1 shows an exploded isometric view of a first embodiment of the device;

FIG. 2 shows a reversed isometric exploded view of the embodiment shown in FIG. 1;

FIG. 3 shows a an exploded isometric view of a preferred embodiment of the device;

FIG. 4A shows an isometric view of the embodiment as shown in FIG. 1; and

FIG. 4B shows a side view of the embodiment shown in FIG. 1;

FIG. 5 illustrates an example device according to one embodiment; and

FIG. 6 illustrates a schematic diagram of a PCB according to one embodiment.

### DETAILED DESCRIPTION OF PREFERRED EMBODIMENTS

### Introduction to Embodiments

Typically, in some embodiments, the device is comprised in a deformable independent format, suitable and comfortable for use subordinate a patient having incontinence potential, in order that a user is enabled to monitor whether the patient has suffered from an incontinence episode. In some embodiments the device may be comprised in a padded cushion, in other embodiments the device may be comprised in an insert for a padded cushion.

Typically the device is for use on a chair, and in other embodiments is configured for use on a bed. As a consequence in some embodiments the padding material comprises cushioning, for example in seat pad embodiments, whereas in other embodiments, such as bed devices, the material comprises a relatively thin material arranged for supporting or protecting apparatus for the means and system.

Preferably only the face is absorbent, wherein all remaining parts are waterproofed and wipeclean for subsequent uses, where the face is machine washable.

In some embodiments the system and means are combined, wherein a colour change is registered locally, wherein at least part of the face is configured to react to fluid for example subject to a change in colour, which colour change is visible remotely. The whole, system and means, may be combined in the flexible material.

In some embodiments the reaction or colour change may be advantageously dislocated from the face, so that such colour change is visible to a remote user even when a patient is necessarily covering some or all of the face in use.

For example the reactive part may be extended into or onto a tab that extends from the face. Typically the reactive part of the face is configured on the upper surface.

Hydro-chromic ink may be used on the face at least in part wherein the ink changes colour in response to recognition of fluid or level of moisture. In this way if a patient or vulnerable person is seated on sleeping on the device, any accidental leakage of urine will prompt a change in colour of the face or part thereof.

Said thermochromic ink may comprise a liquid activated ink formulation comprising: (A) a liquid activated dye, wherein the liquid activated dye is water soluble; (B) a hydrochromic ionic compound; (C) an opacifier; and (D) a solvent.

Advantageously therefore the device is configured to allow users to recognise accidents of incontinence.

In some embodiments the system is electronic and operable or operated using wireless connectivity.

Additionally or alternatively in some embodiments the wireless connectivity allows the device to transmit a signal for alerting a remote user that an accident has occurred.

Such wireless connectivity is typically via radio, infrared or intermachine capability such as Bluetooth® or similar.

The signal is ideally generated by apparatus, wherein such apparatus is embedded in displaceable from the device. The apparatus is protected from damage, wherein the apparatus is waterproofed or located in a waterproof location on the device, for example below or internal to the padding material.

Such signal can be read in preferred embodiments by a smartphone, tablet or personal computer, wherein according to a second aspect of the present invention there is provided a system for monitoring fluid levels on a face of a device such as described herein.

In some embodiments the material is provided with a water resistant covering, which covering may be permanent or displaceable, respectively to wipeclean or replace.

In other embodiments the device material may be absorbent or provided with an absorbent part, wherein the part is replaced, discarded or cleaned after taking on fluid.

Ideally the device includes a module disposed below one surface wherein said surface is arranged in use uppermost. Said module ideally comprises a circuit that is breached or completed by presence of fluid intermediate parts of the circuit.

In some embodiments the module is separate to the apparatus, whereas in preferred embodiments it may be envisaged that the module is contained within and integral to the apparatus.

In some embodiments the apparatus may further include an activation means, which activation means allows activation of the module, and consequently limits energy usage.

Such activation means may comprise a switch, or pressure control, wherein the control activates the apparatus to allow monitoring of fluid levels of the device. In some embodiments such activation means is combined with a timer facility, which timer suspends the apparatus after a period without registering pressure on the activation means.

The apparatus further includes an energy source, which energy source may comprise a wired electrical connection and/or rechargeable or replaceable battery. In embodiments with a battery a battery low level warning may be provided locally, for example a light, or remotely, for example an alert transmitted using wireless connectivity.

The face may be detached form the inner in order that the face may be washed after use. For example the face may comprise a temporary closure mechanism such as a zipper, buttons or press studs.

The module in turn is provided as a wipeclean configuration in order that the module may be used repeatedly. Such module may comprise metallic circuitry which is broken by fluidic contact so as to trigger an alarm signal for remote capture.

In some embodiments the at least part of the face that is reactive to fluid may become transparent or translucent as a reaction to fluid, so as to reveal a colour or pattern below the part.

In other embodiments the at least part of the face that is reactive to fluid may change colour, for example so as to present a different colour or pattern.

In some embodiments the device includes a securement mechanism, configured for securement of the device to a bed, chair or other furniture or location. Such mechanism may comprise a flexible or elastic strapping. Alternatively or additionally such mechanism may comprise hook and loop material such as Velcro®, and/or magnetic portions.

For example in some embodiments the device may be incorporated in or integral to a bed covering such as an undersheet or mattress cover. In such circumstances it may be envisaged that the apparatus comprising active elements of the device, namely the module, remote connectivity and alert, are detachable from the mattress cover or similar, for replacement, maintenance, transport or storage of the device and cleaning, replacement, repair, transport or storage of the mattress cover or similar.

In some embodiments the lower surface is impermeable so as to prevent egress or passage of moisture.

In some other embodiments the lower surface is convex in addition or the alternative, so as to both channel moisture centrally, and allow deformation so as to anchor the device in use.

With reference to the figures there is shown a device for a seat or chair, comprising plural levels namely a lower 2 and upper surface 1, joined about edges to form the face 17, and forming an enclosure that houses padding material 5 and a foil circuit 3 mounted on a flexible sheet 10.

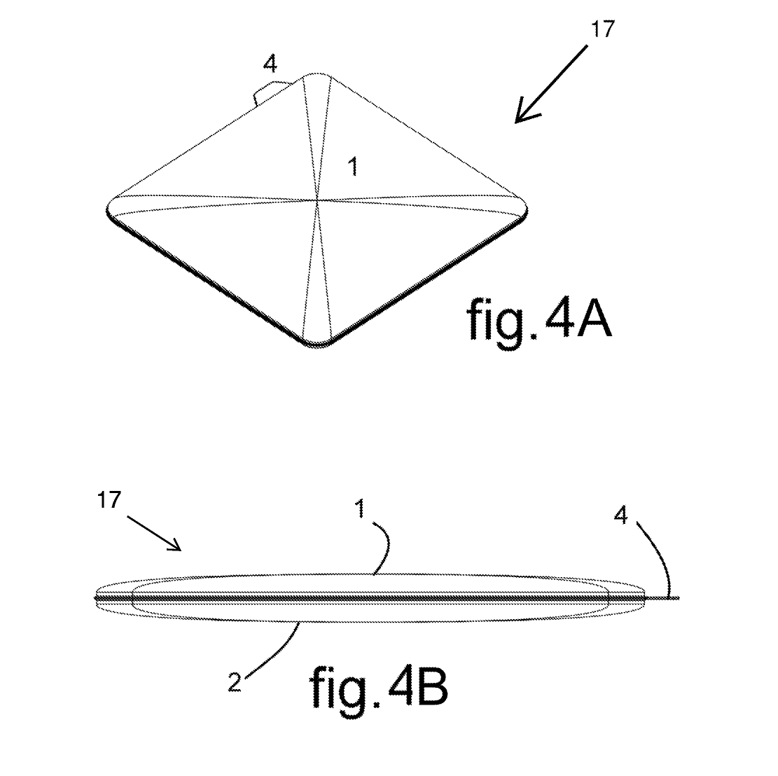

The face 17 may be detached in order that the face may be washed after use. The face 17 comprises a temporary closure mechanism down at least one side between surfaces (not shown) such as a zipper, buttons or press studs. To be sure, the face 17 is comprised of both the upper surface 1 and the lower surface 2 placed together in a mating relationship as illustrated in FIGS. 4A and 4B.

Only the face or upper surface is absorbent, wherein all remaining parts are waterproofed and wipeclean for subsequent uses, where the face is machine washable. To be sure, the upper surface 1 is capable of providing a path for the communication of fluids from outside the device, through the upper surface 1 so as to contact the foil circuit 3. Regardless of the mechanism by which the foil circuit 3 senses fluid contact (either by completing the circuit, shorting the circuit, or alternatively by sensing conductivity changes due to the presence of fluid) the foil circuit 3 will provide a means for sensing the presence of fluid.

The upper surface 1 can be constructed of a fluid permeable member, or at least including a section of material that is fluid permeable. For example, sections, such as section 11 can be made of a fluid permeable membrane or substrate, while ribbing 13 is provided for structural support and flexibility.

In some embodiment, the upper surface 1 is arranged to provide local notification of urinary incontinence and is screen printed with a hydro-chromic white binder, which changes repeatedly from white to transparent when wetted with a fluid and changes back to the original white when dried. In some embodiments a tab 4 is arranged to provide notification alone, so as to provide modesty. Such tab 4 may extend further from the device. Again, the tab 4 allows for visual identification of an incontinence event while the patient is currently sitting or otherwise positioned on the device. Advantageously, the tab 4 can be in fluid communication with a portion of the upper surface 1 that absorbs or permeates fluid, allowing fluid to wick into the tab 4. Thus, the tab 4 can comprise any number of materials that are configured to change color and/or opacity when exposed to fluid.

In other embodiments, the tab 4 extends into the device, below a section of the upper surface 1 that allows for permeation or communication of fluid. Thus, as fluid passes over the tab 4, the tab 4 can react to the fluid and provide a visual indication of fluid presence.

The upper surface 1 faces towards the patient in use, wherein a module 15 is disposed below the upper surface 1. Additionally, the foil circuit 3 and flexible sheet 10 can also be disposed within the device.

In some embodiments, screen printing is used to create portions of the upper surface 1 that comprises a cotton, polyester, Nylon®, blended fabric, or nonwoven fabric. Printing a binder over a design means it is concealed with white in dried condition and appears when the binder becomes transparent with water. The design is ideally appropriate to use.

The upper surface is soft to the touch and excellent in water resistant and rubbing strength so when a colouring agent such as a colouring pigment paste is compounded with the binder and printed, it is reversibly changed from coloured opaque to coloured transparent when coming into contact with fluid.

The foil circuit 3 is connected to the module 15 through flexible wiring 6 having waterproof gaiters (not shown) or other waterproofed connection means, wherein the module comprises a battery 9, printed circuit board (PCB) 8 with a WiFi module or BLE module to provide intermachine operability. In some embodiments, the flexible wiring 6 overlays the flexible sheet 10. Thus, the foil circuit 3 is disposed above the padding material 5 and the module 15 is disposed below the padding material 5.

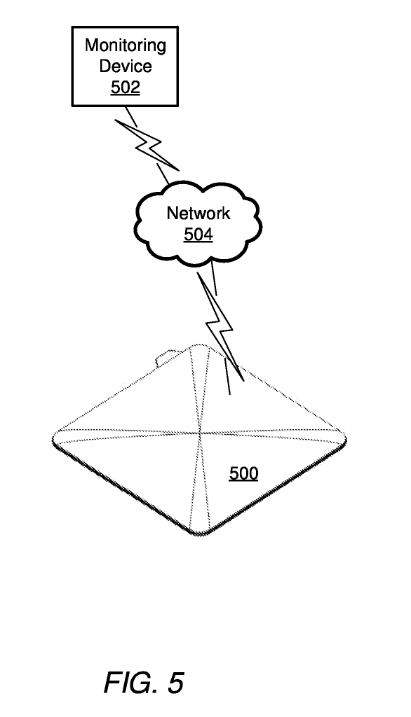

The metallic foil circuit 3 conducts electricity so as to complete a circuit. When moisture is upon (or communicated through) the upper surface 1, the circuit will be broken and a signal sent from the electronic alert module to a server (as illustrated in FIG. 5). This embossed, plastic, moisture-sensing circuit sheet (flexible sheet 10) is dual sided and wipeclean to be easily cleaned with soap and water.

The battery 9 has a universal serial bus connector with a detachable waterproofed cap on a tether. This cap and tether are formed in silicone.

The module may be housed in a rigid housing in some embodiments wherein the pocket 7 is padded for comfort.

The padding material 5 is below the circuit in use, and in some embodiments is water-proof so as to prevent absorption. In other embodiments the padding is disposable after absorption, so as to limit damage to subordinate surfaces or furniture.

The lower surface 2 is provided with a curvature in some embodiments having a substantially rigid form, which form is impermeable and prevents egress of fluid. The curvature of the lower surface 2 directs any fluid present in the device away from the PCB 8 and battery 9 and towards a center of the device.

The lower surface 2 is configured through material selection to flex under weight of a patient, so as to ensure that the device is not liable to movement and consequently is always in use.

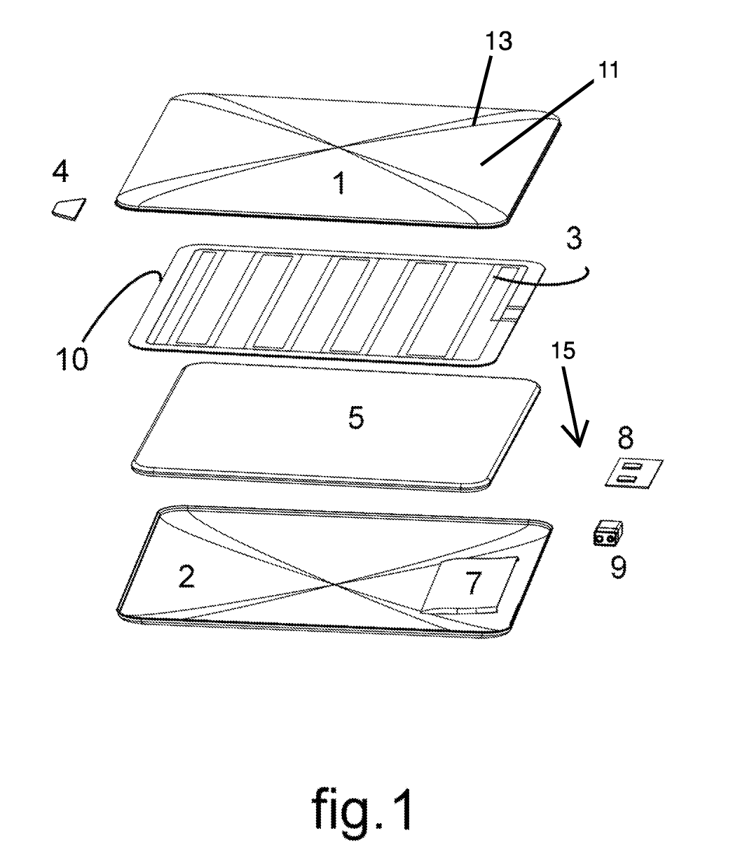

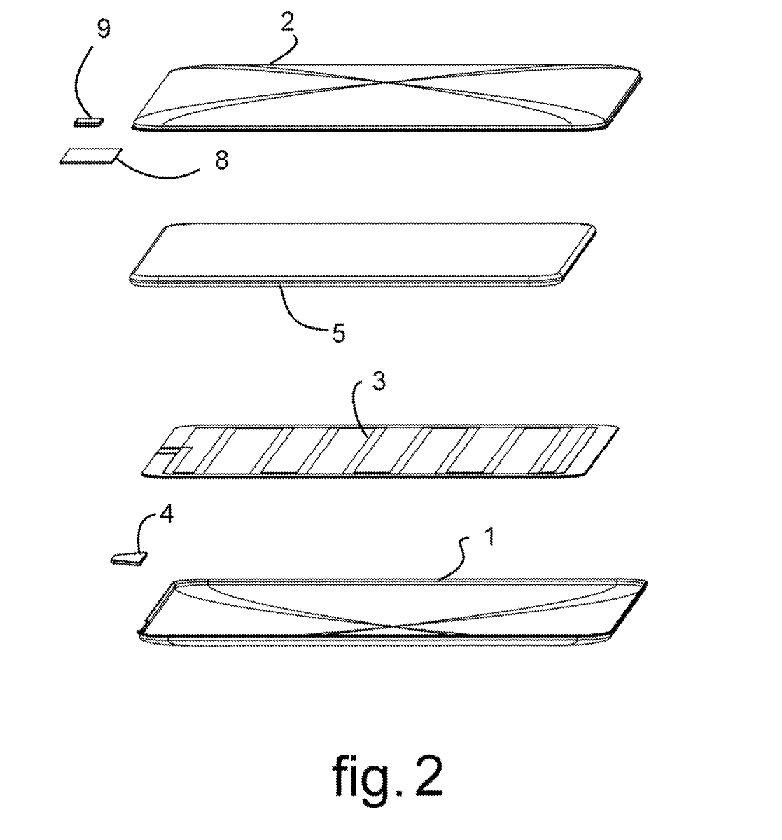

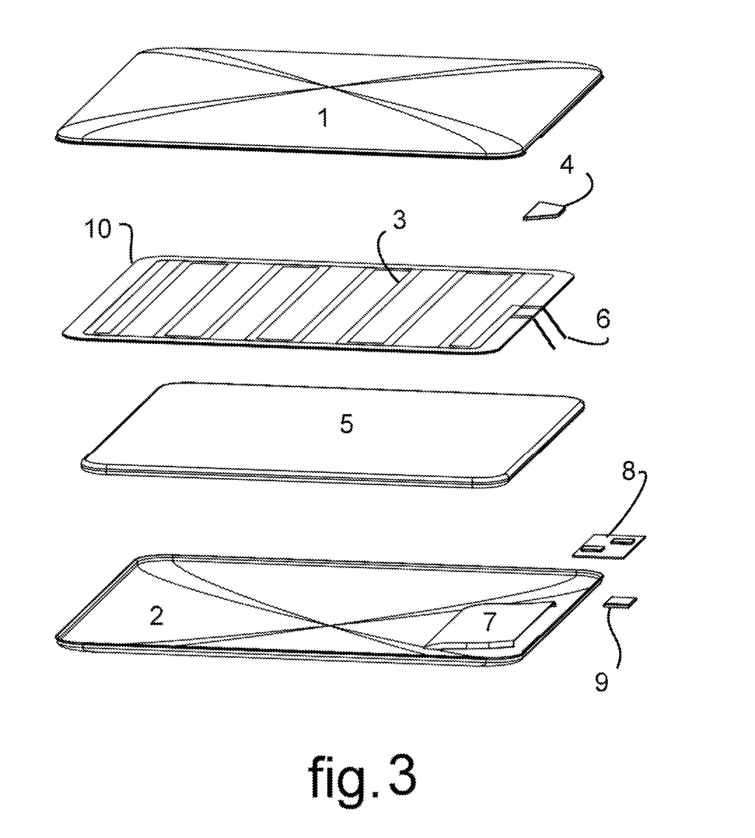

Referring now to FIG. 5, which illustrates an example device 500, such as the device of FIGS. 1-4B. The device 500 can communicatively couple with a monitoring device 502 over a network 504. In some embodiments, the network 504 can include a short range wireless network. In other embodiments, the network 504 can comprise a private and/or public network such as the Internet or a local area network.

Generally, the monitoring device 502 can include any suitable computing device such as a mobile computing device, a personal computer, a server, or the like. The monitoring device 502 is preferably comprised of at least a processor and memory. The memory stores executable instructions that when executed by the processor will serve to display on a display device an indication that fluid has contacted the foil circuit 3. A signal indicative of fluid presence can be generated by the PCB 8, which is comprises a microprocessor that is configured to determine changes in electrical signals output by the foil circuit 3. These changes indicate fluid presence in or on the device 500. In other embodiments, the changes in the electrical signals are determined at the monitoring device 502 when the signals are received from the PCB 8.

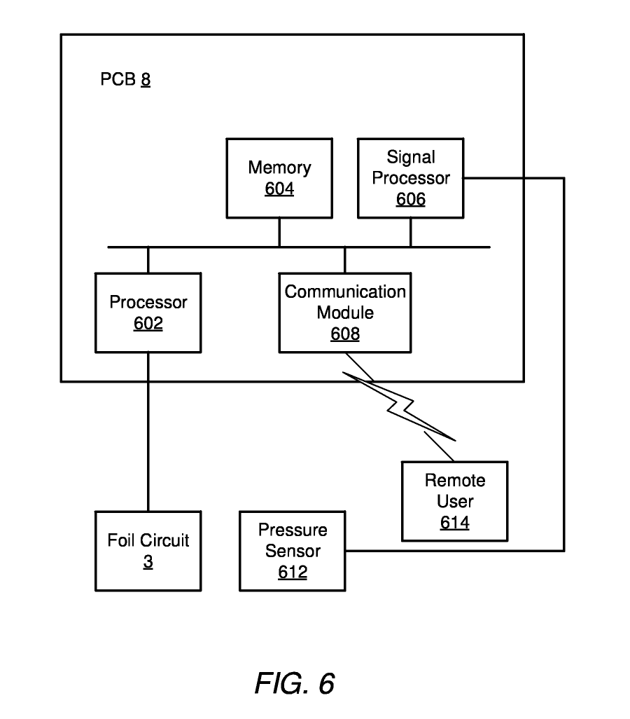

Referring now to FIG. 6, a schematic diagram of a PCB 8 is illustrated. The PCB 8 is communicatively coupled with the foil circuit and a pressure sensor 612, which is utilized to determine the presence of a patient in contact with the device. Broadly, the PCB 8, as mentioned above comprises a processor 602, a memory 604, a signal processing means 606, and a communication module 608. The signal processing means 606 can be configured to interpret signals received from the foil circuit 3 to determine if fluid is present relative to the device. For example, the foil circuit 3 will output a first signal value when no fluid is present and a second signal value when fluid is present. The signal processor 606 is configured to convert the signals received from the foil circuit in such a way that the processor 602 can interpret the signals. For example, if the foil circuit 3 outputs an analog signal, the signal processor 606 can convert the analog signal to a digital signal that can be utilized by the processor 602.

The signal processor 606 can also process signals received from the pressure sensor 612 so that the processor 602 can determine if pressure is being exerted on the device, which may be indicative of patient presence. The processor 602 can also execute instructions such as timing functions, as described above. For example, the processor 602 can selectively disable battery usage when a patient has not used the device within a specified time frame.

The PCB 8 can communicate with a remote user 614 over a network connection as described above using a communication module 608. In some embodiments the communication module 608 is either a short range or long range wireless interface. In other embodiments, the communication module 608 can be a wired connection such as USB or Ethernet.

The present technology has been described by way of examples only and it will be appreciated that variation may be made to the above-mentioned embodiments without departing from the scope of present technology. Firstly it will be understood that any features described in relation to any particular embodiment may be featured in combinations with other embodiments.

With respect to the specification therefore, it is to be realised that the optimum dimensional relationships for the parts of the present technology, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present technology.

Therefore, the foregoing is considered as illustrative only of the principles of the present technology, with variation and implementation obvious and clear on the basis of either common general knowledge or of expert knowledge in the field concerned. Further, since numerous modifications and changes will readily occur to those skilled in the art, it is not desired to limit the present technology to the exact construction and operation shown and described, and accordingly, all suitable modifications and equivalents may be resorted to, falling within the scope of the present technology as set out in the accompanying claims.

1. An incontinence monitoring device comprising a face separated into substantially parallel upper and lower surfaces having a flexible material held therebetween, a means of registering excessive moisture levels with respect to at least one of the surfaces and a system for conveying information regarding the registering of excessive moisture levels to remote users.

2. A device according to claim 1 comprised in a padded cushion.

3. A device according to claim 1 comprised in an insert for a padded cushion.

4-12. (canceled)

13. A device, comprising:
a face separated into substantially parallel upper and lower surfaces;
means of detecting a presence of fluid with respect to at least one of the surfaces; and
means for conveying information regarding the registering of excessive moisture levels to remote users.

14. The device according to claim 13, wherein the means of detecting a presence of fluid with respect to at least one of the surfaces comprises a foil circuit mounted on a flexible sheet, the foil circuit being in communication with the least one of the surfaces such that when the fluid contacts the least one of the surfaces the foil circuit is completed or breached to provide an electrical output that is different from an output of the foil circuit when the fluid is not present.

15. The device according to claim 14, further comprising a microprocessor in electrical communication with the foil circuit, the microprocessor sensing a change in the electrical conductivity of the foil circuit so as to detect a completion or breach of the foil circuit caused by the presence of the fluid.

16. The device according to claim 13, further comprising a tab that is in fluid communication with the upper surface, the tab providing a visual indication when the fluid contacts the upper surface.

17. The device according to claim 16, wherein the tab comprises a hydrochromic, opacifier, or solvent that provide the visual indication.

18. The device according to claim 15, further comprising means for activating the microprocessor.

19. The device according to claim 18, wherein the means for activating the microprocessor are based on presence, or lack thereof, of a user in contact with the device.

20. The device according to claim 13, wherein the means for conveying information regarding the registering of excessive moisture levels to remote users comprises a wired or wireless communication module that transmits a signal generated by the microprocessor that indicates that the fluid has contacted or permeated the upper surface.

21. The device according to claim 13, further comprising a padding material that supports the means of detecting a presence of fluid with respect to at least one of the surfaces.

22. The device according to claim 21, further comprising a module disposed below the padding material, the module comprising a microprocessor and a communication module.

23. The device according to claim 22, further comprising a pocket formed on an inside surface of the lower surface, wherein the module is disposed within the pocket.

24. The device according to claim 13, wherein the means of detecting a presence of fluid with respect to at least one of the surfaces comprises an embossed, plastic, moisture-sensing circuit sheet.

25. A device, comprising:
an upper surface and a lower surface that cooperate to form an enclosure, wherein the upper surfaces is at least partially fluid permeable;
a moisture-sensing circuit disposed within the enclosure; and
a module communicatively coupled with the moisture-sensing circuit, the moisture-sensing circuit outputting a signal to a remove user when fluid passes through or contacts the upper surface, and contacts the moisture-sensing circuit.

26. The device according to claim 25, wherein the lower surface is concave and fluid contacting the lower surface is directed inward towards a center of the lower surface.

27. The device according to claim 26, wherein the module is disposed in a pocket mounted to the lower surface, wherein the pocket is positioned away from the center of the lower surface.

28. The device according to claim 25, wherein the upper surface comprises a soft material having a coloring agent that reversibly changes between opaque and transparent when the fluid is present.

29. A system, comprising:
a device comprising:
an upper surface and a lower surface that cooperate to form an enclosure, wherein the upper surfaces is at least partially fluid permeable;
a moisture-sensing circuit disposed within the enclosure; and
a module communicatively coupled with the moisture-sensing circuit, the moisture-sensing circuit outputting a signal to a remove user when fluid passes through or contacts the upper surface, and contacts the moisture-sensing circuit; and
a computing device communicatively coupled with the device over a network, the computing device being configured to monitoring fluid levels on the upper surface of the device.
